(19) United States
(12) Patent Application Publication
(10) Pub. No.: US 2021/0047645 A1
(43) Pub. Date: Feb. 18, 2021

(54) METHODS FOR TREATING OVARIAN CANCER

(71) Applicant: Mayo Foundation for Medical Education and Research, Rochester, MN (US)

(72) Inventors: Scott H. Kaufmann, Rochester, MN (US); Saravut J. Weroha, Rochester, MN (US); Cordelia D. McGehee, Rochester, MN (US); Rachel M. Hurley, Rochester, MN (US); Annapoorna Venkatachalam, Rochester, MN (US); Andrea E. Wahner Hendrickson, Rochester, MN (US); Jamie Bakkum-Gamez, Byron, MN (US)

(21) Appl. No.: 16/978,815

(22) PCT Filed: May 8, 2019
(86) PCT No.: PCT/US2019/031272

§ 371 Date: Sep. 8, 2020

Related U.S. Application Data
(60) Provisional application No. 62/668,508, filed on May 8, 2018.

(51) Int. Cl. C12N 15/113; A61P 35/00

(52) U.S. Cl. CPC C12N 15/1135; C12N 2310/531; C12N 2310/14; A61P 35/00

(57) ABSTRACT

Materials and methods for using minicircles against ovarian cancer mRNA targets, to prevent metastasis, are provided herein.

### CROSS-REFERENCE TO RELATED APPLICATIONS

This application claims the benefit of U.S. Patent Application Serial No. 62/668,508, filed on May 8, 2018. The disclosure of the prior application is considered part of (and is incorporated by reference in) the disclosure of this application.

### TECHNICAL FIELD

This document relates to materials and methods for using minicircles against ovarian cancer mRNA targets in order to reduce the risk of or prevent metastasis.

### BACKGROUND

Ovarian cancer is a common cancer in women in the United States. With a case fatality rate of about 70%, ovarian cancer is the deadliest gynecological malignancy, and one of the deadliest cancers overall. The high fatality rate reflects the fact that there is no screening test available, the symptoms and signs are nonspecific, and the disease most often is diagnosed in advanced stages when it has spread throughout the peritoneal cavity. Two classes of drugs, anti-angiogenic antibodies and poly ADP ribose polymerase (PARP) inhibitors, have been approved and used for treating ovarian cancer, but these agents only affect progression-free survival and have not been shown to impact overall survival. Thus, there remains a critical need for improved ovarian cancer therapies.

### SUMMARY

This document is based, at least in part, on the development of methods for using minicircles encoding therapeutic small oligonucleotides directed against ovarian cancer mRNA targets, in order to reduce the risk of (or prevent) metastasis. In some cases, as described herein, minicircles can be delivered intraperitoneally after an ovarian tumor is surgically removed, but prior to closing the incision. Advantages of using minicircle-based methods to deliver therapeutic small oligonucleotides (e.g., shRNAs or siRNAs) against ovarian cancer targets include, for example, increased transfection efficiency, lack of a cellular or immune response, and persistent expression.

In a first aspect, this document features a method for treating a mammal having cancer. The method can include administering to the mammal a pharmaceutical composition containing one or more minicircles that encode an RNA component targeted to an RNA molecule associated with ovarian cancer, where the RNA component, when expressed in a cell, reduces expression of the targeted RNA molecule. The mammal can be a human. The RNA component can include a small interfering RNA (siRNA). The RNA component can include a short hairpin RNA (shRNA). The administering can include introducing the composition into a peritoneal cavity of the mammal after surgical resection of an ovarian tumor, but before closing the surgical incision. The RNA molecule associated with ovarian cancer can encode MYC, CCNE1, BCLXL, MCL1, or RB1. The RNA molecule associated with ovarian cancer can include an mRNA encoding a polypeptide involved in tumor implantation and growth on the outside of the bowel (e.g., EGFL6, FABP4, GPAT3, POSTN, FAP, EPYC, or PAPPA). The RNA molecule associated with ovarian cancer can include an mRNA encoding a driver or enabler of ovarian cancer growth (e.g., mutant PI3KCA, mutant KRAS, MUC16, SIK2, MAP2K6, SMG1, UPF1, TNF, TRAF2, USPS, FERMT2, PARD6B, MECOM, ARID2 in ARID4-mutant tumors, AKT2, or AKT3). The RNA molecule associated with ovarian cancer can include an mRNA encoding a polypeptide that contributes to ovarian cancer stem cell maintenance or self-renewal (e.g., CD133 (PROM1), ALDH1A1, ALDH1A2, PAX8, PRKCI, LGR6, LGR5, or KRT6B). The RNA molecule associated with ovarian cancer can include an mRNA encoded by a DNA repair gene (e.g., a DNA repair gene encoding XRCC2, MCMI, FOXM1, BRD4, WEE1, POLE, FAAP24, ATR, or BRD9). The RNA molecule associated with ovarian cancer can include an mRNA encoding an immunosuppressive polypeptide (e.g., CD274, CTLA4, IDO1, TIM3 (HAVCR2), LAG3, or a CT45A family member). The RNA molecule associated with ovarian cancer can include an mRNA encoding a drug resistance protein (e.g., ABCB1 or ABCG2). The composition can contain two or more different minicircles, where each minicircle encodes an RNA component targeted to a different RNA molecule associated with ovarian cancer. The composition can contain three to five different minicircles, where each minicircle encodes an RNA component targeted to a different RNA molecule associated with ovarian cancer. The method can further include selecting the one or more minicircles in the composition based on a genomic analysis of the mammal. The method can further include monitoring the mammal for cancer recurrence.

In another aspect, this document features a minicircle encoding an RNA component targeted to an RNA molecule associated with ovarian cancer, wherein the RNA component, when expressed in a cell, reduces expression of the targeted RNA molecule. The RNA component can include an siRNA. The RNA component can include an shRNA. The RNA molecule associated with ovarian cancer can encode MYC, CCNE1, BCLXL, MCL1, or RB1. The RNA molecule associated with ovarian cancer can include an mRNA encoding a polypeptide involved in tumor implantation and growth on the outside of the bowel (e.g., EGFL6, FABP4, GPAT3, POSTN, FAP, EPYC, or PAPPA). The RNA molecule associated with ovarian cancer can include an mRNA encoding a driver or enabler of ovarian cancer growth (e.g., mutant PI3KCA, mutant KRAS, MUC16, SIK2, MAP2K6, SMG1, UPF1, TNF, TRAF2, USPS, FERMT2, PARD6B, MECOM, ARID2 in ARID4-mutant tumors, AKT2, or AKT3). The RNA molecule associated with ovarian cancer can include an mRNA encoding a polypeptide that contributes to ovarian cancer stem cell maintenance or self-renewal (e.g., CD133 (PROM1), ALDH1A1, ALDH1A2, PAX8, PRKCI, LGR6, LGR5, or KRT6B). The RNA molecule associated with ovarian cancer can include mRNA encoded by a DNA repair gene (e.g., a DNA repair gene encoding XRCC2, MCMI, FOXM1, BRD4, WEE1, POLE, FAAP24, ATR, or BRD9). The RNA molecule associated with ovarian cancer can include an mRNA encoding an immunosuppressive polypeptide (e.g., CD274, CTLA4, IDO1, TIM3 (HAVCR2), LAG3, or a CT45A family member). The RNA molecule associated with ovarian cancer can include an mRNA encoding a drug resistance protein (e.g., ABCB1 or ABCG2).

In another aspect, this document features a pharmaceutical composition containing a pharmaceutically acceptable carrier and one or more minicircles encoding an RNA component targeted to an RNA molecule associated with ovarian cancer, wherein the RNA component, when expressed in a cell, reduces expression of the targeted RNA molecule. The RNA component can include an siRNA. The RNA component can include an shRNA. The RNA molecule associated with ovarian cancer can encode MYC, CCNE1, BCLXL, MCL1, or RB1. The RNA molecule associated with ovarian cancer can include an mRNA encoding a polypeptide involved in tumor implantation and growth on the outside of the bowel (e.g., EGFL6, FABP4, GPAT3, POSTN, FAP, EPYC, or PAPPA). The RNA molecule associated with ovarian cancer can include an mRNA encoding a driver or enabler of ovarian cancer growth (e.g., mutant PI3KCA, mutant KRAS, MUC16, SIK2, MAP2K6, SMG1, UPF1, TNF, TRAF2, USPS, FERMT2, PARD6B, MECOM, ARID2 in ARID4-mutant tumors, AKT2, or AKT3). The RNA molecule associated with ovarian cancer can include an mRNA encoding a polypeptide that contributes to ovarian cancer stem cell maintenance or self-renewal (e.g., CD133 (PROM1), ALDH1A1, ALDH1A2, PAX8, PRKCI, LGR6, LGR5, or KRT6B). The RNA molecule associated with ovarian cancer can include mRNA encoded by a DNA repair gene (e.g., a DNA repair gene encoding XRCC2, MCMI, FOXM1, BRD4, WEE1, POLE, FAAP24, ATR, or BRD9). The RNA molecule associated with ovarian cancer can include an mRNA encoding an immunosuppressive polypeptide (e.g., CD274, CTLA4, IDO1, TIM3 (HAVCR2), LAG3, or a CT45A family member). The RNA molecule associated with ovarian cancer can include an mRNA encoding a drug resistance protein (e.g., ABCB1 or ABCG2). The composition can contain two or more different minicircles, where each minicircle encodes an RNA component targeted to a different RNA molecule associated with ovarian cancer. The composition can contain three to five different minicircles, where each minicircle encodes an RNA component targeted to a different RNA molecule associated with ovarian cancer. The pharmaceutically acceptable carrier can be selected from the group consisting of water, saline, a binding agent, a filler, a lubricant, a disintegrate, and a wetting agent.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the description below. Other features, objects, and advantages of the invention will be apparent from the description, and from the claims.

### DETAILED DESCRIPTION

Ovarian cancer often disseminates throughout the peritoneal cavity, but not beyond. Thus, at the time of recurrence, the disease often remains confined (or largely confined) to the peritoneal cavity. In fact, a large majority of ovarian cancer deaths result from bowel obstruction due to recurrence in the peritoneal cavity. Thus, therapies that can prevent or delay recurrence in the abdomen and pelvis are likely to have a major impact on quality of life and survival of women with this disease. In addition, according to current therapy protocols, many women with newly diagnosed ovarian cancer undergo extensive surgery followed by multiple cycles of chemotherapy with a platinum agent (e.g., carboplatin) and an antimitotic agent (e.g., paclitaxel). The extent of disease left behind at the end of surgery can have a major impact on time to progression after chemotherapy; if all visible disease is surgically removed, the time to recurrence averages two to three years, whereas patients who have visible disease remaining after surgery (typically ranging from 0 to 1 cm in diameter) are more likely to experience recurrence within 12 to 18 months.

This document provides materials and methods for diminishing the extent of disease in the perioperative or early postoperative period, which may improve survival. For example, in some embodiments, this document provides methods for using minicircles to treat the surgical field at the time of ovarian cancer resection, or shortly thereafter. Such treatment can reduce or even prevent the growth of unresected cancer, thereby delaying or preventing recurrence. In some cases, minicircles can be administered, with or without chemotherapy, into the peritoneal cavity at the time of recurrence. Minicircles (also referred to as “minivectors”) are circular plasmid derivatives can range in size from about 225 base pairs (bp) to about 5000 bp (e.g., about 250, about 300, about 350, about 400, about 500, about 600, about 700, about 800, about 900, about 1000, about 1500, about 2000, about 2500, about 3000, about 3500, about 4000, about 4500, or about 5000 bp, or about 225 to about 300 bp, about 300 to about 400 bp, about 400 to about 500 bp, about 500 to about 600 bp, about 600 to about 700 bp, about 700 to about 800 bp, about 800 to about 900 bp, about 900 to about 1000 bp, about 1000 to about 1500 bp, about 1500 to about 2000 bp, about 2000 to about 2500 bp, about 2500 to about 3000 bp, about 3000 to about 3500 bp, about 3500 to about 4000 bp, about 4000 to about 4500 bp, or about 4500 to about 5000 bp), that typically are free from prokaryotic vector parts. Minicircles can be used as transgene carriers for the genetic modification of mammalian cells, with the advantage that they are not likely to be perceived as foreign and destroyed since they do not contain bacterial DNA sequences. The relatively small size of minicircles also can extend their cloning capacity and can facilitate their delivery into cells.

Minicircle preparation typically includes two steps: (1) production of a “parental” bacterial plasmid with eukaryotic inserts in E. coli, and (2) induction of a site-specific recombinase while the parental plasmid is still in the bacteria (Nehlsen et al., Gene Ther. Mol. Biol. 10:233-244, 2006; and Kay et al., Nature Biotechnol. 28:1287-1289, 2010). These steps can be followed by excision of prokaryotic vector parts using recombinase target sequences at both ends of the insert, and recovery of the resulting minicircle by, for example, capillary gel electrophoresis. In some cases, minicircles can lack an origin of replication, so they do not replicate within target cells and will eventually disappear as the cell divides. Nonviral, self-replicating minicircles can be developed, however; these can replicate due to the presence of a scaffold/matrix attachment region (S/MAR) element (Broil et al., J. Mol. Biol. 395:950-965, 2010; Argyros et al., J. Mol. Med. 89:515-529, 2011; Heinz et al., CliniBook-Nonviral Platform, Clinigene Network: 271-277, 2010; and Nehlsen et al., Minicircle and Plasmid DNA Vectors-The Future of Non-Viral and Viral Gene-Transfer, Schleef, M. (ed.), Wiley-VCH, Weinheim, pp. 115-162, 2013).

The minicircles to be used in the methods described herein can encode, for example, one or more shRNAs, one or more siRNAs, one or more long non-coding RNAs (RNAs that typically are longer than 200 nucleotides), one or more microRNAs, or one or more guide RNAs for a clustered regularly-interspaced short palindromic repeat (CRISPR) Cas system, where the encoded RNAs are targeted to various mRNAs/genes. Short hairpin RNAs (shRNAs) and short interfering RNAs (siRNAs) can be RNA interference (RNAi) tools that can be used for short-term silencing of selected genes (see, e.g., Moore et al., Methods Mol. Biol. 629:141-158, 2010). After introduction into cells, the minicircles can make siRNA or shRNA as long as they persist within the cells. In some cases, the minicircles can persist in cells for weeks to months (e.g., one to two weeks, two to four weeks, four to six weeks, six to eight weeks, two to three months, three to six months, or longer than six months). Thus, in some embodiments, minicircle-encoded siRNAs or shRNAs can affect metastasis of cancer cells (e.g., by impacting pathways that can discourage metastasis) until other processes intervene and the cancer cells are killed.

A siRNA can be a synthetic RNA duplex designed to specifically target a particular mRNA for degradation. siRNAs can be used to knock down genes in a variety of cell lines that are amenable to transfection with synthetic oligonucleotides. siRNAs can consist of two RNA strands that typically form a duplex about 19 to 25 base pairs in length, with 3′ dinucleotide overhangs. siRNAs can be generated through solid-phase chemical synthesis methods (e.g., 2′-ACE chemistry). siRNAs can be transfected into cells via cationic lipid or polymer-based transfection reagents, via electroporation, via viral vectors, or via chemical modifications that can be added to the siRNA duplex to aid in cellular uptake. Once inside the cell, a siRNA can elicit gene silencing by binding to its target mRNA, thereby targeting the mRNA for cleavage and degradation.

shRNAs also can be used for mRNA silencing, but shRNAs typically are synthesized within cells by DNA vector-mediated production. For example, cells can be transfected with a plasmid or infected with a viral vector encoding a shRNA. Thus, while siRNA duplexes are delivered directly to the cytosol, shRNAs can integrate into the cellular DNA. shRNAs can consist of two complementary RNA sequences, each about 19 to 22 nucleotides in length, that are linked by a short loop of about 4 to 11 nucleotides. After transcription, the shRNA sequence can be exported to the cytosol, where it is processed into an siRNA duplex that can bind to its target mRNA, triggering target-specific mRNA degradation.

In some cases, the mRNAs/genes to be targeted according to the methods provided herein can include genes that play roles in ovarian cancer biology. Such genes include, without limitation, MYC (a transcription factor that is involved in proliferation), CCNE1 (a cell cycle regulator), BCL2L1 (which encodes the protein BCLXL, a regulator of apoptosis), MCL1 (a regulator of apoptosis that has a second role as a component of the electron transport chain), and RB1 (a tumor suppressor that often is mutated or disrupted in ovarian cancer). In some cases, however, the shRNAs/siRNAs can be targeted to mRNAs that play less obvious roles in ovarian cancer biology, either alone or in combination with siRNAs/shRNAs targeted to one or more of the genes listed above. For example, targets for the minicircle-encoded siRNAs/shRNAs can include mRNAs encoding proteins involved in tumor implantation and growth on the outside of the bowel (e.g., EGFL6, FABP4, GPAT3, POSTN, FAP, EPYC, and PAPPA), mRNAs encoding drivers and enablers of ovarian cancer growth (e.g., mutant PI3KCA, mutant KRAS, MUC16, SIK2, MAP2K6, SMG1, UPF1, TNF, TRAF2, USPS, FERMT2, PARD6B, MECOM, ARID2 in ARID4-mutant tumors, AKT2, and AKT3), mRNAs encoding proteins that contribute to ovarian cancer stem cell maintenance and self-renewal (e.g., CD133 (PROM1), ALDH1 (such as the ALDH1A1 and ALDH1A2 isoforms), PAX8, PRKCI, LGR6, LGR5, and KRT6B), mRNAs encoding selected DNA repair genes (e.g., XRCC2, MCMI, FOXM1, BRD4, WEE1, POLE, FAAP24, ATR, and BRD9), mRNAs encoding immunosuppressive proteins (e.g., CD274, CTLA4, IDO1, TIM3 (HAVCR2), LAG3, and CT45A family members, including but not limited to CT45A1, CT45A2, and CT45A3), and mRNAs encoding drug resistance proteins (e.g., ABCB1 and ABCG2). GENBANK® accession numbers for each of these targets are provided in TABLE 1.

TABLE 1

| Official | GENBANK ® | |
|---|---|---|
| Gene Symbol | Chromosomal | |
| (Human Gene | Reference | |
| Organization | Location | |
| name) | (GRCh38.p12) | Range |
| MYC | NC_000008.11 | 127735434 . . . 127742951 |
| CCNE1 | NC_000019.10 | 29811994 . . . 29824317 |
| BCL2L1 | NC_000020.11 | 31664452 . . . 31723999, complement |
| MCL1 | NC_000001.11 | 150574551 . . . 150579738 |
| RB1 | NC_000013.11 | 48303747 . . . 48481890 |
| EGFL6 | NC_000023.11 | 13569575 . . . 13633575 |
| FABP4 | NC_000008.11 | 81478497 . . . 81483238, complement |
| GPAT3 | NC_000004.12 | 83535634 . . . 83605875 |
| POSTN | NC_000013.11 | 37562582 . . . 37598844, complement |
| FAP | NC_000002.12 | 162170684 . . . 162243557, complement |
| EPYC | NC_000012.12 | 90963679 . . . 91005026, complement |
| PAPPA | NC_000009.12 | 116153752 . . . 116402321 |
| PI3KCA | NC_000003.12 | 179148114 . . . 179240093 |
| KRAS | NC_000012.12 | 25204789 . . . 25251003, complement |
| MUC16 | NC_000019.10 | 8848840 . . . 9010390, complement |
| SIK2 | NC_000011.10 | 111602391 . . . 111726917 |
| MAP2K6 | NC_000017.11 | 69414697 . . . 69553861 |
| SMG1 | NC_000016.10 | 18804853 . . . 18926428, complement |
| UPF1 | NC_000019.10 | 18831405 . . . 18868232 |
| ARID2 | NC_000012.12 | 45729706 . . . 45908040 |
| AKT2 | NC_000019.10 | 40230317 . . . 40285531, complement |
| AKT3 | NC_000001.11 | 243488233 . . . 243851079, complement |
| PROM1 | NC_000004.12 | 15968226 . . . 16084059, complement |
| ALDH1A1 | NC_000009.12 | 72900662 . . . 72953317, complement |
| ALDH1A2 | NC_000015.10 | 57953424 . . . 58065923, complement |
| PAX8 | NC_000002.12 | 113215997 . . . 113278921, complement |
| PRKCI | NC_000003.12 | 170222432 . . . 170305982 |
| LGR6 | NC_000001.11 | 202193990 . . . 202319761 |
| LGR5 | NC_000012.12 | 71439770 . . . 71586310 |
| KRT6B | NC_000012.12 | 52446651 . . . 52452126, complement |
| XRCC2 | NC_000007.14 | 152646498 . . . 152676165, complement |
| MCM7 | NC_000007.14 | 100092728 . . . 100101940, complement |
| FOXM1 | NC_000012.12 | 2857680 . . . 2877155, complement |
| BRD4 | NC_000019.10 | 15236836 . . . 15332543, complement |
| WEE1 | NC_000011.10 | 9573681 . . . 9589767 |
| ATR | NC_000003.12 | 142449235 . . . 142578826, complement |
| BRD9 | NC_000005.10 | 863735 . . . 892824, complement |
| CD274 | NC_000009.12 | 5450503 . . . 5470567 |
| CTLA4 | NC_000002.12 | 203867788 . . . 203873960 |
| IDO1 | NC_000008.11 | 39913809 . . . 39928790 |
| TIM3 (HAVCR2) | NC_000005.10 | 157085832 . . . 157109237, complement |
| LAG3 | NC_000012.12 | 6772483 . . . 6778455 |
| CT45A1 | NC_000023.11 | 135708398 . . . 135723318 |
| ABCB1 | NC_000007.14 | 87503863 . . . 87713323, complement |
| FAAP24 | NC_000019.10 | 32972212 . . . 32977495 |
| TRAF2 | NC_000009.12 | 136881933 . . . 136926615 |
| FERMT2 | NC_000014.9 | 52857271 . . . 52951097, complement |
| PARD6B | NC_000020.11 | 50731544 . . . 50753741 |
| MECOM | NC_000003.12 | 169083499 . . . 169663781, complement |
| USP5 | NC_000012.12 | 6852121 . . . 6866632 |
| POLE | NC_000012.12 | 132623758 . . . 132687524, complement |
| ABCG2 | NC_000004.12 | 88090264 . . . 88231626, complement |
| TNF | NC_000006.12 | 31575567 . . . 31578336 |
| CT45A2 | NC_000023.11 | 135811979 . . . 135820062, complement |
| CT45A3 | NC_000023.11 | 135760067 . . . 135768224, complement |

The effectiveness of a minicircle targeting a particular gene can be evaluated using one or more models of ovarian cancer. These models include, for example, patient-derived xenograft (PDX) models that reflect the biology of ovarian cancer in many different ways, including the retention of characteristic genomic features (e.g., mutations, rearrangements, and/or copy number alterations), biology (e.g., the presence or absence of ascites, and/or the propensity to attach to the exterior of the bowel), and chemotherapy sensitivity. Such models can have a strong correlation between response of the source patient to carboplatin/paclitaxel therapy and response of the tumorgraft grown in immunosuppressed mice to the same agents. Various PDX models are available from the John Weroha lab at the Mayo Clinic, and have been characterized by extensive genomic analysis, including panel testing for frequently mutated genes in ovarian cancer, RNA sequencing, whole exome sequencing, and deep proteomics. The PDX models thus provide a useful system for assessing the impact of minicircle-mediated changes in gene expression ex vivo and in vivo under various conditions (e.g., newly injected tumor, fully established tumor, and with or without chemotherapy).

This document also provides methods for identifying shRNA and/or siRNA sequences that can be used in the treatment methods described herein. The methods of identification can include any of the following components:
assessing the expression of particular siRNA or shRNA sequences from minicircles and evaluating their efficacy of mRNA/gene knockdown, as well as killing of cells (e.g., ovarian cancer cell lines) in vitro;
assessing the impact of siRNA/shRNA-expressing minicircles on cell lines and/or PDXs when grown as spheroids/organoids ex vivo, with endpoints of three-dimensional growth and target downregulation;
assessing the impact of siRNA/shRNA-expressing minicircles on the growth of tumor cells (e.g., ovarian cancer PDXs or cell lines) in vivo when the minicircles are administered intraperitoneally with tumor cells or at various time points after tumor cell delivery (e.g., a day later, 3 days later, a week later, two weeks later, and/or a month later), using endpoints of implantation and tumor growth;
assessing the impact of siRNA/shRNA-expressing minicircles on PDXs when the minicircles are administered alone to established PDXs, using endpoints of target gene expression and PDX time to growth;
assessing the impact of siRNA/shRNA-expressing minicircles in PDXs when the minicircles are administered with and/or after conventional chemotherapy (e.g., carboplatin/paclitaxel, gemcitabine, liposomal doxorubicin, or PARP inhibitors) to established PDXs, following both target gene expression and PDX time to growth; and
assessing the impact of siRNA/shRNA-expressing minicircles on wound healing in patients or laboratory animals after laparotomy and possibly end-to-end bowel anastomosis.

In addition, the use of minicircles to deliver siRNAs/shRNAs can be evaluated via biodistribution studies to assess whether minicircles delivered to the peritoneal cavity are confined to cells within the peritoneal cavity, and to determine how many layers of cells the minicircles penetrate. Such studies can be carried out by administering minicircles labeled with a tag (e.g., digoxygenin) to laboratory animals (e.g., mice) and then sacrificing animals to perform immunohistochemistry at periodic intervals, or by administering a minicircle encoding a particular marker (e.g., GLP-1) that typically is not expressed or is only expressed at very low levels in the animals, and then sacrificing animals at various time points and staining for the marker. Retention experiments and toxicological experiments also can be carried out using these methods.

Minicircles encoding siRNAs/shRNAs can be incorporated into compositions for administration to a subject (e.g., a human or a non-human mammal with cancer). Any appropriate methods for formulating and subsequently administering therapeutic compositions can be performed. For example, minicircles as described herein can be admixed, encapsulated, conjugated or otherwise associated with other molecules, molecular structures, or mixtures of compounds such as, for example, liposomes, receptor or cell targeted molecules, microvesicles, or other formulations for assisting in uptake, distribution and/or absorption.

In some embodiments, a composition can contain one or more minicircles in combination with a pharmaceutically acceptable carrier. Pharmaceutically acceptable carriers include, without limitation, pharmaceutically acceptable solvents, suspending agents, and other pharmacologically inert vehicles for delivering, e.g., nucleic acids to a subject. Pharmaceutically acceptable carriers can be liquid or solid, and can be selected with the planned manner of administration in mind so as to provide for the desired bulk, consistency, and other pertinent transport and chemical properties, when combined with one or more therapeutic compounds and any other components of a given pharmaceutical composition. Typical pharmaceutically acceptable carriers include, without limitation: water; saline solution; binding agents (e.g., polyvinylpyrrolidone or hydroxypropyl methylcellulose); fillers (e.g., lactose or dextrose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

In some embodiments, a pharmaceutical composition as provided herein can include a sterile aqueous solution (e.g., sterile physiological saline), which also can contain one or more buffers, diluents, or other suitable additives (e.g., penetration enhancers, carrier compounds and other pharmaceutically acceptable carriers). Pharmaceutical compositions include, but are not limited to, solutions, emulsions, aqueous suspensions, microvesicles, and liposome-containing formulations. These compositions can be generated from a variety of components that include, for example, preformed liquids, self-emulsifying solids and self-emulsifying semisolids.

Formulations containing one or more minicircles also can contain other adjunct components that may conventionally be found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions, such as dyes, preservatives, antioxidants, opacifiers, thickening agents, and stabilizers. Further, the compositions can be mixed with auxiliary agents, e.g., lubricants, preservatives, stabilizers, wetting agents, emulsifiers, salts for influencing osmotic pressure, buffers, colorings, penetration enhancers, and aromatic substances. When added, however, such materials should not unduly interfere with the biological activities of the other components within the compositions.

Any appropriate technique can be used to prepare a pharmaceutical formulation containing one or more minicircles as disclosed herein. Such techniques include the step of bringing into association the active ingredients (e.g., minicircles) with the desired pharmaceutical carrier(s). Typically, the formulations can be prepared by uniformly and intimately bringing the active ingredients into association with liquid carriers or finely divided solid carriers or both. Formulations can be sterilized if desired, provided that the method of sterilization does not interfere with the effectiveness of the molecules(s) contained in the formulation.

This document also provides methods for administering one or more minicircles encoding siRNAs/shRNAs to cancer patients after surgical resection of an ovarian tumor, to knock down expression of targets such as those listed herein. For example, a composition containing one or more different minicircles (e.g., one, two, three, four, five, six, seven, eight, nine, ten, or more than ten minicircles), each encoding a siRNA/shRNA directed to a different mRNA/gene target, can be administered into the peritoneal cavity of a patient before the incision is closed. In some embodiments, one or more minicircles can be dissolved in a volume of fluid (e.g., 100 ml to 250 ml, 250 ml to 500 ml, 500 ml to 1 L, or 1 L to 2 L of a fluid such as normal saline or Ringer's lactate), and the fluid can be infused into the peritoneal cavity (e.g., at room temperature or warmed to a temperature such as 37° C. or 42° C.). The composition can be allowed to dwell in the abdomen for a selected length of time (e.g., 10 to 30 minutes, 30 to 60 minutes, 60 to 120 minutes, or 120 to 240 minutes) and then removed. In some cases, a composition containing one or more minicircles can be dripped or sprayed into the peritoneal cavity (e.g., onto a tumor bed). In some cases, the methods provided herein can include administering two or more (e.g., three, four, five, or more than five) minicircles targeted to a single gene. In some cases, a combination of two or more (e.g., three, four, five, or more than five) minicircles targeting different genes can be administered.

It is to be noted that the methods described herein are intended to encompass personalized medicine approaches. In some cases, for example, the choice of mRNAs to be targeted, either singly or in combination, can be based on knowledge of the genomic alterations in the ovarian cancer of a particular patient. Thus, the therapeutic methods described herein can be coupled to genomic analysis of a cancer in a patient. Based on the genomic analysis, a combination of minicircles can be administered to a patient as a custom-tailored cocktail that includes one to ten (e.g., one to three, two to four, three to five, four to six, five to seven, six to eight, seven to nine, or eight to ten) different minicircles selected from a menu of options (e.g., 15 to 20 options). For example, a method for treating CCNE1 amplified ovarian cancer (which can be particularly difficult to treat with chemotherapy) can include administering minicircles encoding CCNE1 shRNA and BCL2L1 shRNA (for those with BCLXL overexpression), CCNE1 shRNA and MCL1 shRNA (for those with MCL1 overexpression), or CCNE1 shRNA with

PAX8 shRNA and/or PRKCI shRNA (for those with PRKCI overexpression). Thus, treatment can be personalized based on which genes are amplified or overexpressed in a particular patient, if such information is known at the time of minicircle administration. Accordingly, the methods provided herein can include assessing a tumor within a patient to determine which genes should be targeted, in an attempt to increase the likelihood of a beneficial effect.

The invention will be further described in the following example, which does not limit the scope of the invention described in the claims.

### EXAMPLE

Minicircle combinations are selected for administration to a mammal with an ovarian tumor based on genomics of the tumor and/or gene expression profiling. For example, mammals having ovarian cancers with CCNE1 overexpression are identified and treated with CCNE1 shRNA-containing minicircles. Based on other gene expression patterns, additional minicircles are added to the treatment. Thus, when BCLXL expression is elevated, BCL2L1 shRNA is administered in addition to the CCNE1 shRNA; if MYC expression is amplified, MYC shRNA is administered in addition to the CCNE1 shRNA, and if PRKCI expression is amplified, PRKCI shRNA is administered in addition to the CCNE1 shRNA. The shRNAs encoded by the minicircles are targeted to regions of the selected gene(s) that are unique to those gene(s), to achieve selective knockdown.

The efficacy of combinations of minicircles targeted to various genes is monitored by assessing the expression of the targeted genes/proteins using methods such as quantitative RT-PCR, immunoblotting, ELISA, and immunohistochemistry on biopsy samples, and/or by assessing effects on ovarian cancer growth and progression using methods such as radiological imaging (e.g., x-ray, ultrasound, computed tomography, PET scan, or a combination thereof) following the growth of target lesions. In some cases, the efficacy of minicircle treatment is assessed by testing a body fluid sample (e.g., blood, plasma, or serum) to evaluate the expression of a tumor marker such as CA-125 (the product of the MUC16 gene) or another marker of disease burden, such as the amount of cell-free circulating DNA that can be attributed to an ovarian cancer origin. In addition, in some cases the efficacy of minicircle treatment is evaluated by determining the extent of tumor-associated mutations or gene rearrangements that can be detected in cell-free circulating DNA.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating a mammal having cancer, comprising administering to the mammal a pharmaceutical composition comprising one or more minicircles encoding an RNA component targeted to an RNA molecule associated with ovarian cancer, wherein the RNA component, when expressed in a cell, reduces expression of the targeted RNA molecule.

2. The method of claim 1, wherein the mammal is a human.

3. The method of claim 1, wherein the RNA component comprises a small interfering RNA (siRNA).

4. The method of claim 1, wherein the RNA component comprises a short hairpin RNA (shRNA).

5. The method of claim 1, wherein the administering comprises introducing the composition into a peritoneal cavity of the mammal after surgical resection of an ovarian tumor, but before closing the surgical incision.

6-18. (canceled)

19. The method of claim 1, wherein the composition comprises two or more different minicircles, and wherein each minicircle encodes an RNA component targeted to a different RNA molecule associated with ovarian cancer.

20. The method of claim 1, wherein the composition comprises three to five different minicircles, and wherein each minicircle encodes an RNA component targeted to a different RNA molecule associated with ovarian cancer.

21. The method of claim 1, further comprising selecting the one or more minicircles in the composition based on a genomic analysis of the mammal.

22. The method of claim 1, further comprising monitoring the mammal for cancer recurrence.

23. A minicircle encoding an RNA component targeted to an RNA molecule associated with ovarian cancer, wherein the RNA component, when expressed in a cell, reduces expression of the targeted RNA molecule.

24. The minicircle of claim 23, wherein the RNA component comprises an siRNA.

25. The minicircle of claim 23, wherein the RNA component comprises an shRNA.

26-38. (canceled)

39. A pharmaceutical composition comprising a pharmaceutically acceptable carrier and one or more minicircles encoding an RNA component targeted to an RNA molecule associated with ovarian cancer, wherein the RNA component, when expressed in a cell, reduces expression of the targeted RNA molecule.

40. The pharmaceutical composition of claim 39, wherein the RNA component comprises an siRNA.

41. The pharmaceutical composition of claim 39, wherein the RNA component comprises an shRNA.

42-54. (canceled)

55. The pharmaceutical composition of claim 39, wherein the composition comprises two or more different minicircles, and wherein each minicircle encodes an RNA component targeted to a different RNA molecule associated with ovarian cancer.

56. The pharmaceutical composition of claim 39, wherein the composition comprises three to five different minicircles, and wherein each minicircle encodes an RNA component targeted to a different RNA molecule associated with ovarian cancer.

57. The pharmaceutical composition of claim 39, wherein the pharmaceutically acceptable carrier is selected from the group consisting of water, saline, a binding agent, a filler, a lubricant, a disintegrate, and a wetting agent.
